(19) United States
(12) Patent Application Publication
(10) Pub. No.: US 2015/0140039 A1
(43) Pub. Date: May 21, 2015

(54) IMMUNOLOGICAL MARKERS FOR ADJUVANT THERAPY IN MELANOMA

(71) Applicant: John Wayne Cancer Institute, Santa Monica, CA (US)

(72) Inventors: Dave S.B. Hoon, Los Angeles, CA (US); Donald L. Morton, Pacific Palisades, CA (US); Connie G. Chiu, Santa Monica, CA (US); Kelly Chong, Santa Monica, CA (US)

(21) Appl. No.: 14/401,076

(22) PCT Filed: May 16, 2013
(86) PCT No.: PCT/US13/41448

§ 371 Date: Nov. 13, 2014

Related U.S. Application Data
(60) Provisional application No. 61/648,066, filed on May 16, 2012.
(60) Provisional application No. 61/647,679, filed on May 16, 2012.

(51) Int. Cl. C12Q 1/68; A61K 39/04

(52) U.S. Cl. CPC C12Q 1/6886; A61K 39/04; C12Q 2600/156; C12Q 2600/106; A61K 2039/585
USPC 424/248.1; 435/6.11; 506/7; 506/9; 506/2; 506/12

(57) ABSTRACT

The present invention concerns embodiments for determining whether an individual in need of immunotherapy will be responsive to the immunotherapy. Determination of one or more SNPs in particular genes is predictive of responsiveness to immunotherapy, particularly in individuals that have melanoma, for example. In certain embodiments, the SNPs are in ITGB2, SP1 1O, ILIB, IL23R, SLC11A1, IL12B, CCR5, TNF, ILIO, CXCL12, BTNL2, ANKRD20A4, CD 14, P2X7, IL8, TLR2, and/or CD209.

This application claims priority to U.S. Provisional Patent Application Ser. No. 61/647,679, filed May 16, 2012 and also to U.S. Provisional Patent Application Ser. No. 61/648,066, filed May 16, 2012, both of which applications are incorporated by reference herein in their entirety.

### STATEMENT REGARDING FEDERALLY SPONSORED RESEARCH OR DEVELOPMENT

This invention was made with government support under Grant Numbers CA76489 and CA012582 awarded by the National Cancer Institute. The government has certain rights in the invention.

### TECHNICAL FIELD

The field of the invention includes at least cell biology, molecular biology, immunology, and medicine, including cancer medicine.

### BACKGROUND

Melanoma is an immunogenic cancer, whereby adjuvant immunotherapy has shown some success. However, there are no effective immune biomarker tests to identify patients most likely to benefit from immunotherapy. Current immune-modulating anti-cancer treatment include: Bacillus Calmette-Guérin (BCG), interferon (IFN), interleukin-2 (IL-2), and monoclonal antibody-based treatment such as ipilimumab (MDX-010, MDX-101; Yervoy® Bristol-Myers Squibb), vemurafenib (RG7204, PLX4032; Zelboraf®, Genentech), sorafenib (Nexavar®, Bayer and Onyx Pharmaceuticals), anti-PD1/PD1-ligand therapy, dabrafenib, and tumor-infiltrating lymphocyte therapies. BCG therapy is a form of immunomodulation, and serves as a model for testing and confirmation of immune biomarkers. Single nucleotide polymorphisms are specific gene signatures embodied by a person. The present invention provides predictive clinical effect of a single nucleotide polymorphism(SNP) immune biomarker panel in melanoma patients treated with immunotherapy, using BCG therapy as a model. The SNP markers chosen represent molecules involved in a range of human immunologic function.

### BRIEF SUMMARY

In embodiments of the invention, there are one or more SNPs that are biomarkers for immunotherapy responsiveness in an individual being treated for cancer. The immunotherapy may be of any kind, including cancer vaccine, therapeutic antibodies, cell-based immunotherapy, or methods for immumosuppression of the microenvironment.

In embodiments of the invention, there is a novel immunological biomarker SNP panel in BCG adjuvant therapy in individuals with melanoma. In specific embodiments, the SNP panel includes two or more SNPs, three or more SNPs, four or more SNPs, five or more SNPs, six or more SNPs, seven or more SNPs, eight or more SNPs, or nine or more SNPs, ten or more SNPs, eleven or more SNPs, twelve or more SNPs, thirteen or more SNPs, fourteen or more SNPs, fifteen or more SNPs, sixteen or more SNPs, seventeen or more SNPs, eighteen or more SNPs, nineteen or more SNPs, twenty or more SNPs, twenty-one or more SNPs, twenty two or more SNPs, twenty three or more SNPs, twenty four or more SNPs, twenty five or more SNPs, twenty six or more SNPs, or twenty seven or more SNPs. In some embodiments, multiple SNPs are assayed to determine individuals receiving BCG immunotherapy with a favorable disease outcome. In specific embodiments, there is a 28-SNP panel that indicates that there is an exceptionally favorable disease outcome in patients receiving immunotherapy. In certain embodiments, there is a 9-SNP panel that indicates that there is a favorable outcome in patient receiving immunotherapy. In particular embodiments, the panel represents a stratifying predictive biomarker panel for identifying patients that are inherently responsive to BCG and other immunomodulating or immune-related agents.

The gene identity of the SNPs may be found in any suitable database including, for example, from the hg19 Human Genome Browser from the UCSC Bioinformatics site or from the National Institute of Biotechnology Information's GenBank® database. Any of 28 SNPs that identified individuals that are responsive to BCG immunotherapy are encompassed in the invention, but in specific embodiments the SNPs are located in one or more of ITGB2, SP110, IL1B, IL23R, SLC11A1, IL12B, CCR5, TNF, IL10, CXCL12, BTNL2, ANKRD20A4, CD14, P2X7, IL8, TLR2, and CD209. One of skill in the art recognizes that particular SNPs from these genes are identified respectively in Table 1. The SNPs may be located in coding sequences, non-coding sequences, regulatory regions, introns, promoters, and the like.

Although in specific embodiments the methods and compositions are utilized with an individual having metastatic melanoma, in some embodiments the methods and compositions are utilized with an individual having stage I, II, III, or IV melanoma. In specific embodiments, the methods and compositions are utilized with an individual having metastatic melanoma (American Joint Cancer Commission, AJCC, Stage II, III and IV). Although in particular embodiments the SNPs related to the invention herein are useful for identifying particular subgroups of individuals with melanoma, in alternative embodiments the SNPs described herein are useful for identifying particular subgroups of individuals with any other type of cancer, such as breast, prostate, brain, colorectal, liver, blood, kidney, thyroid, pancreatic, esophageal, testicular, cervical, ovarian, gall bladder, and so forth.

In at least some cases, the metatstatic melanoma has been resected, whereas in other cases the melanoma has not been resected. In embodiments of the invention, a primary tumor has been resected.

The melanoma of the individual can originate in any part of the body including skin, acral, mucosal, or the eye. In some embodiments of the invention, an individual receiving the BCG agent and in need of determination of one or more SNPs related to the invention may have any type of melanoma, although in specific embodiments the individual has cutaneous, lentigo maligna, acral lentiginous, mucosal, occular, or desmoplastic. In certain embodiments of the invention, an individual receiving the immunotherapy agent and in need of determination of a multi-SNP panel (for example, of 2, 3, 4, 5, 6, 7, 8, 9, 10, 11, 12, 13, 14, 15, 16, 17, 18, 19, 20, 21, 22, 23, 24, 25, 26, 27 or 28 SNPs) related to the invention may have any type of melanoma.

In some embodiments of the invention, one or more SNPs related to the invention are useful for prediction of disease-free survival, melanoma-specific survival, and/or overall survival.

In particular embodiments, one or more characterstics of an individual are considered for prognosis or diagnosis of individuals with melanoma in addition to the SNPs of the invention, such as age, sex, Breslow depth (tumor thickness in millimeters), ulceration, number of positive lymph nodes, and primary site.

In specific embodiments, one or more nucleic acids are assayed in one or more samples from an individual for one or more particular SNPs. Following analysis of the sample(s) for the presence of one or more SNPs associated with the invention, it is determined whether or not the individual is responsive to BCG immunotherapy or another immunomodulating agent in melanoma, and in specific embodiments, will have favorable outcome for the immunotherapy. In specific embodiments, the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, or CD209. In some cases, two or more SNPS from one of the aforementioned genes is utilized in analysis.

The melanoma may be treated or may have been treated by other means of cancer therapy, including one or more of surgery, radiation, chemotherapy, imiquimod therapy, and/or immunomodulating therapy, such as with Bacillus Calmette-Guérin (BCG), interferon (IFN), interleukin-2 (IL-2) and monoclonal antibody-based treatment such as ipilimumab, vemurafenib, sorafenib, and anti-PD1/PD1-ligand therapy, dabrafenib, tumor-infiltrating lymphocyte therapies or other melanoma targeted therapies or vaccine related therapies, for example. Cytokines or monoclonal Ab therapy may be employed, in particular cases.

In some embodiments, there is a method of determining whether or not an individual will be responsive to Bacillus Calmette-Guérin (BCG) immunotherapy, comprising the step of detecting the presence or absence and/or allelic configuration of one or more single nucleotide polymorphisms (SNPs) in nucleic acid from a sample from the individual, wherein when the one or more SNPs are present in the nucleic acid, the individual will respond to the BCG immunotherapy. In specific embodiments, the individual has melanoma, such as Stage III melanoma. In certain aspects, the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209.

In certain embodiments, the method comprises detecting the presence or absence of a SNP in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209.

Individuals subjected to methods of the invention may have received or are receiving a melanoma therapy, such as one that comprises surgery, radiation, chemotherapy, or immunotherapy, for example. Individuals subjected to the methods of the invention may be a mammal, such as human. Samples obtained from the individual may comprise tissue biopsy, and in some cases the method further comprises the step of obtaining the sample from the individual. DNA and/or RNA may be isolated from the sample. In some cases, at least part of the nucleic acid from the sample is amplified prior to detection.

In some embodiments, there is a method of determining whether or not an individual will be responsive to an immunotherapy, comprising the step of detecting the presence or absence and/or allelic configuration of one or more single nucleotide polymorphisms (SNPs) in nucleic acid from a sample from the individual, wherein when the one or more SNPs are present in the nucleic acid, the individual will respond to the BCG immunotherapy, wherein the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209. In certain embodiments, the method comprises detecting the presence or absence of a SNP in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209. Individuals subjected to methods of the invention may have received or are receiving a melanoma therapy, such as one that comprises surgery, radiation, chemotherapy, or immunotherapy, for example. Individuals subjected to the methods of the invention may be a mammal, such as human. Samples obtained from the individual may comprise tissue biopsy, and in some cases the method further comprises the step of obtaining the sample from the individual. DNA and/or RNA may be isolated from the sample. In some cases, at least part of the nucleic acid from the sample is amplified prior to detection.

In some embodiments, there is a method of treating an individual for cancer, wherein the cancer treatment comprises an immunomodulating agent, comprising the steps of providing an anti-cancer agent to the individual; and providing an immunomodulating agent to the individual upon detection of the presence of one or more SNPs in genes selected from the group consisting of ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209; and a combination thereof. In specific embodiments, the immunomodulating agent comprises BCG immunotherapy. In certain aspects, the method further comprises the step of identifying an individual in need of the cancer treatment. The cancer may be melanoma, such as stage III melanoma. In some embodiments, the individual has had surgical removal of at least part of the melanoma.

In some embodiments, there is a method of treating melanoma in a patient comprising obtaining information on the presence or absence of one or more single nucleotide polymorphisms (SNPs) in nucleic acid from a sample from the patient, wherein when the one or more SNPs are present in the nucleic acid, the individual will respond to Bacillus Calmette-Guérin (BCG) immunotherapy; and administering BCG immunotherapy to the patient. In specific aspects, the individual has Stage III melanoma. In other specific aspects, the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209.

In embodiments of the invention, there is a method of determining whether or not an individual will be responsive to immunotherapy, comprising the step of a) detecting the presence and/or absence and/or allelic configuration of one or more single nucleotide polymorphisms (SNPs) in nucleic acid from a sample from the individual, wherein when the one or more SNPs are respectively present or absent or has a particular allelic configuration in the nucleic acid, the individual will be responsive to the immunotherapy, or b) detecting a representative value in relation to a pre-determined threshold value for a group of the SNPs in nucleic acid from a sample from the individual, wherein when the representative value is greater than the threshold, the individual will respond to the immunotherapy, wherein the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209.

In embodiments of the invention, there is a method of determining whether or not an individual will be responsive to immunotherapy, comprising the step of providing a representative value for a specific allelic configuration in one or more single nucleotide polymorphisms (SNPs) in nucleic acid from a sample from the individual and comparing the representative value to a pre-determined threshold value, wherein when the representative value is greater than the threshold, the individual will respond to the immunotherapy, wherein the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209. The pre-determined threshold value may be determined by incorporating one or more variables into principal component analysis (PCA). PCA is standard in the art. In specific embodiments, at least one of the SNPs is identified in Table 1. In specific embodiments, the SNPs include all of the SNPs identified in Table 1. In particular embodiments, the immunotherapy comprises Bacillus Calmette-Guérin (BCG), cytokine therapy, monoclonal antibody-based therapy, anti-PD1/PD1-ligand therapy, MAPKinase inhibitor, anti-toll like receptor (TLR) therapy, dabrafenib, tumor-infiltrating lymphocyte therapies, or a combination thereof. In certain embodiments, the cytokine therapy comprises interferon (IFN), interleukin-2 (IL-2) therapy, or a combination thereof. In some embodiments, the monoclonal antibody-based treatment comprises ipilimumab, vemurafenib, sorafenib, or a combination thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS AND TABLES

For a more complete understanding of the present invention, reference is now made to the following descriptions taken in conjunction with the accompanying drawing, in which:

FIG. 1 shows results of principal components analysis (PCA) using the Stage IV MMAIT patients (BCG-treated group). The first two principal components are shown.

FIG. 2 shows results of PCA using the Stage IV MMAIT patients (BCG-treated group). The first two principal components are shown. A cutoff (red-coloured line) at the mean of component 2 was taken to separate patients into two groups: Group 1 is Comp2>0.126; Group2 is Comp2<=0.126

FIGS. 3A-3F show application of a formula (and cutoff above 0.126). A Kaplan-Meier curve was generated for the study patients, presented by AJCC Stage. OS=overall survival (FIGS. 3A, 3C, and 3E). DFS=disease-free survival (FIGS. 3B, 3D, and 3F). MMAIT=BCG-treated group. MSLT (sentinel lymph node positive patients no adjuvant therapy post surgery (=non-treated group (i.e. control group).

Table 1 shows the allele state of each of the 28 SNPs in the invention.

Table 2 shows the coefficients used for the calculation of principal component 2.

Table 3 shows multivariate Cox proportional hazards regression. The PCA grouping is shown to be an independent predictor of survival among MMAIT (melanoma vaccine treated patients with BCG) patients.

### DETAILED DESCRIPTION

As used herein the specification, “a” or “an” may mean one or more. As used herein in the claim(s), when used in conjunction with the word “comprising”, the words “a” or “an” may mean one or more than one. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

Throughout this application, the term “about” is used to indicate that a value includes the standard deviation of error for the device or method being employed to determine the value.

### I. GENERAL EMBODIMENTS

Embodiments concern the determination of whether or not an individual will be responsive to an adjuvant cancer immunotherapy. An individual is considered responsive to cancer immunotherapy when upon use of the immunotherapy the individual has an improvement of the cancer (including reduced proliferation of cancer or killing of cancer cells) and/or has an increase in disease-free survival, melanoma-specific survival, or overall survival, for example. General embodiments of the invention employ detection of one or more SNPs in genes selected from the group consisting of ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209. Upon determination of the presence and/or specific allelic configuration of the one or more SNPs, an action may be taken. For example, when one or more of the SNPs is detected, the individual may receive the cancer immunotherapy, but when one or more of the SNPs is not detected, the individual may not receive the cancer immunotherapy.

In specific embodiments of the invention, there is determination of a representative value in relation to a pre-determined threshold value for a group of SNPs, and it is this representative value that is indicative whether or not an individual will be responsive to the immunotherapy. The threshold and the value may be determined by standard means in the art, including by using principal components analysis that utilizes not only the genotype of a SNP but other variables, such as age, primary site, gender, and so forth.

In embodiments of the invention, the presence of one or more SNPs in the 28-SNP panel is scored according to the invention formula for each SNP (Table 2). The final “treatment” score is dichotomized into “responders to immunotherapy” (score >0.126) or “non-responders to immunotherapy” (score <0.126). In the clinical setting, a blood sample is obtained from an individual. The sample is analyzed for the presence of one or more SNPs and the profile subjected to scoring according to an invention formula. The result of the final score (i.e. “responders to immunotherapy” vs. “non-responders to immunotherapy”) aids medical providers in decision to treat with immunotherapy or to seek alternate therapy.

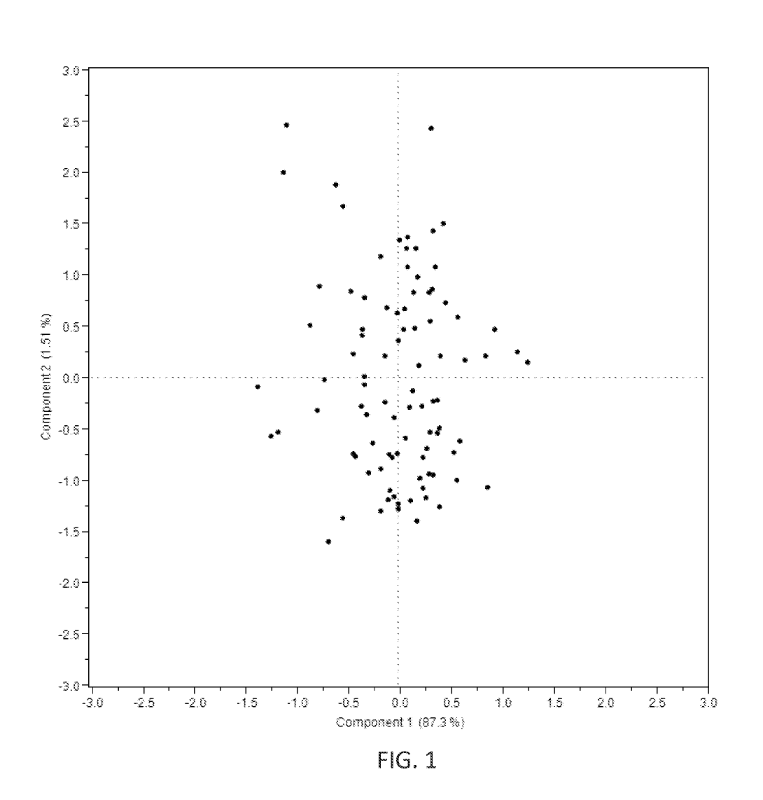

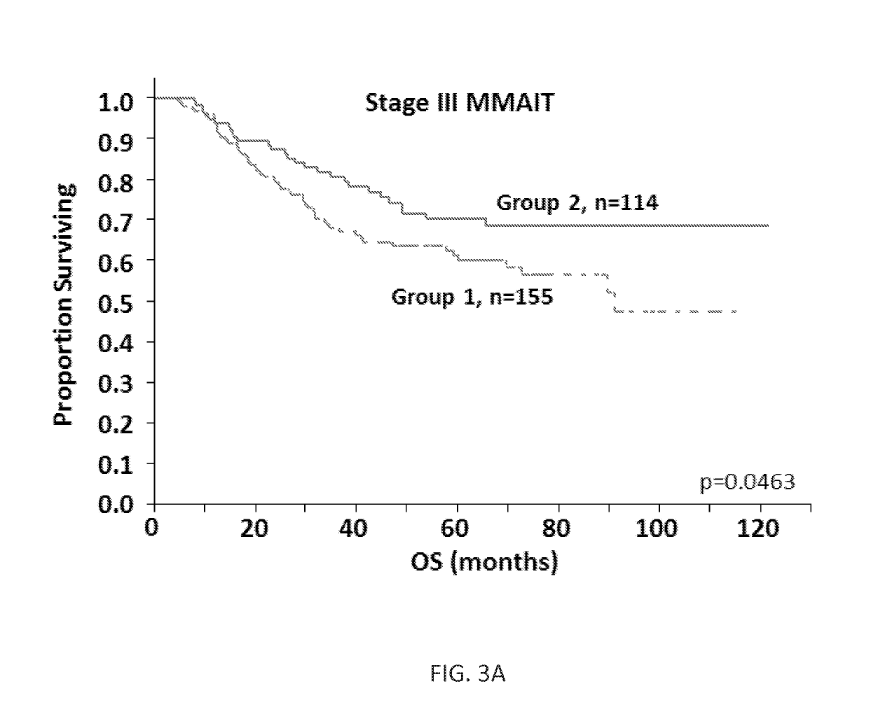

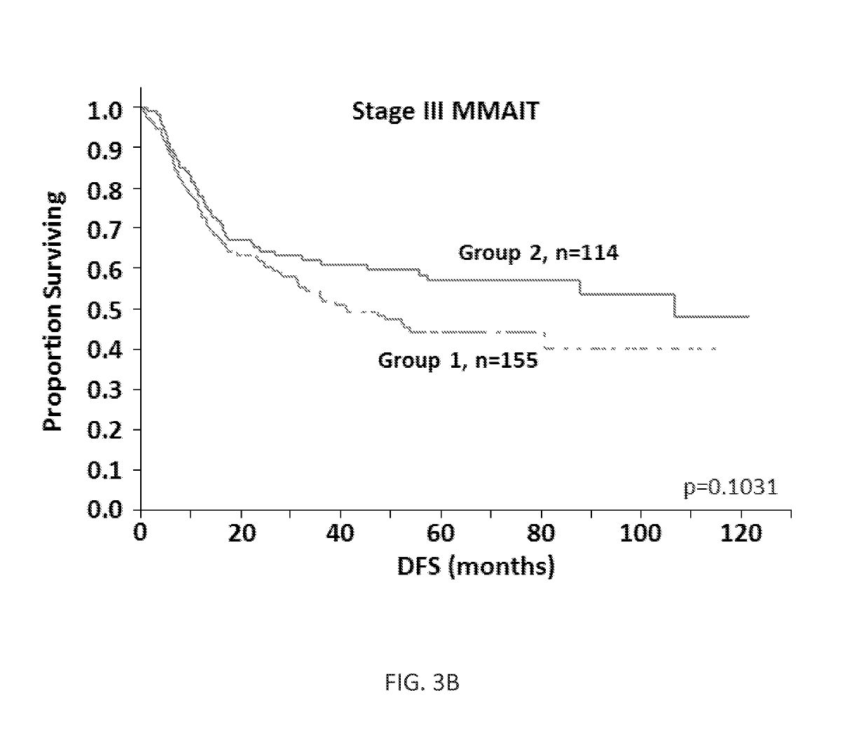

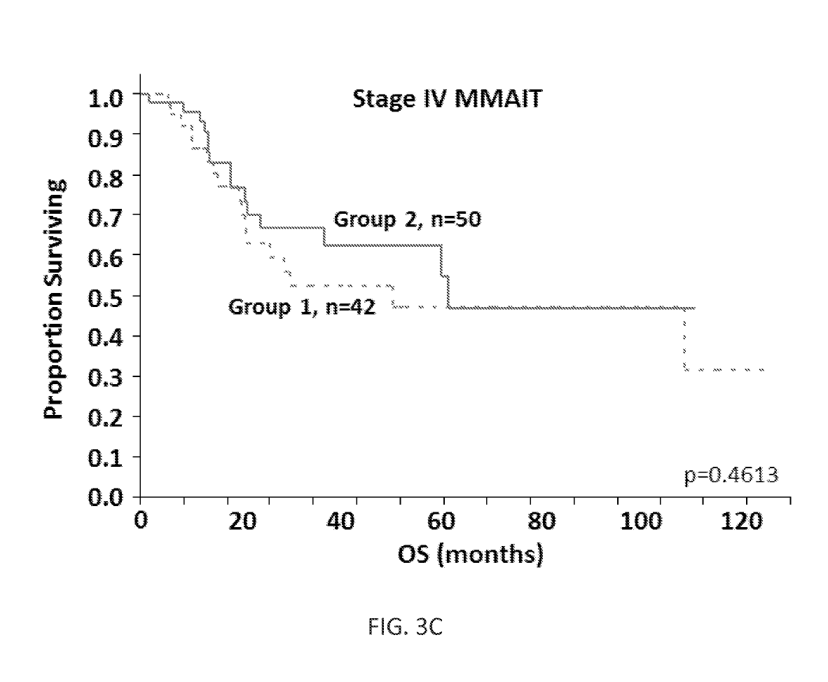

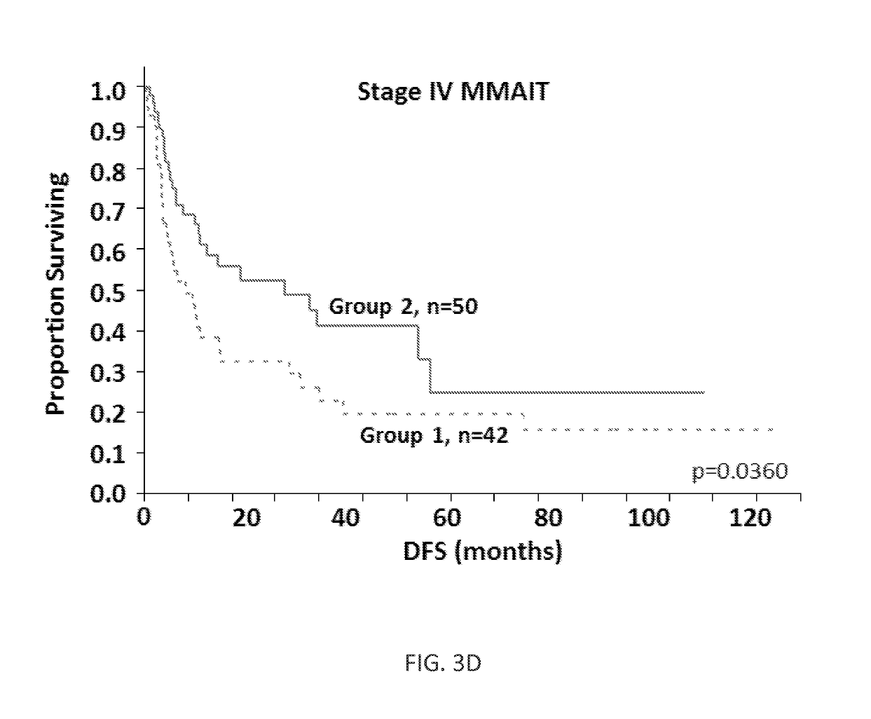

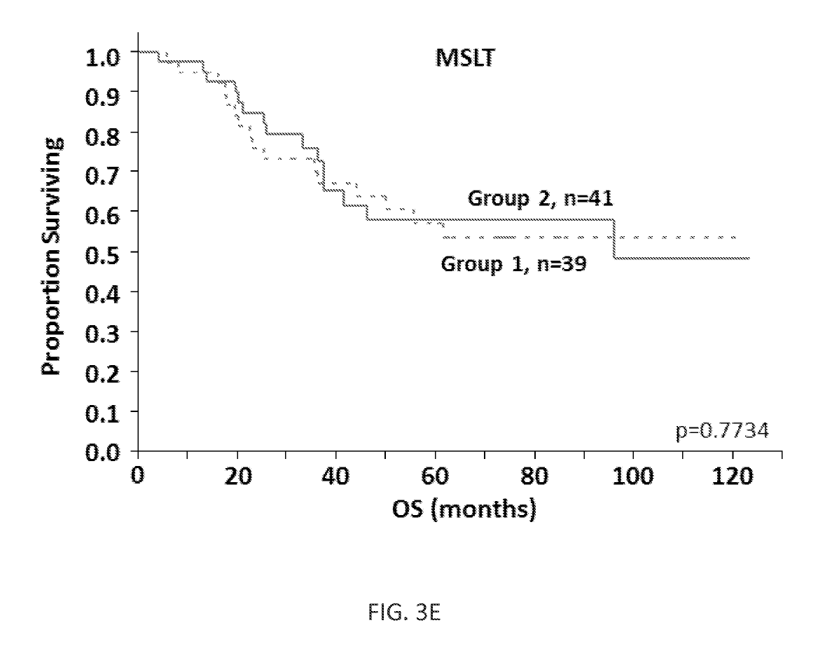

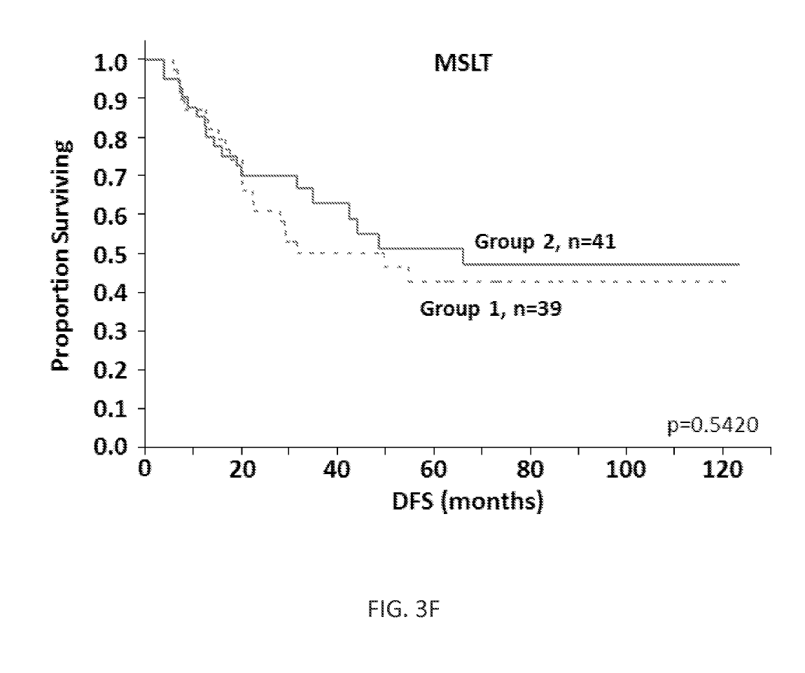

An exemplary procedure for identifying whether an individual will be responsive to a therapy is provided herein. In specific cases, an individual's sample, such as a blood sample, is tested for the allelic configuration at one or more particular SNP sites. A particular allelic pair is given a score (from 0 to 3; see Table 1). For example, for 3UTR122GA, the number of “A” alleles were counted—if the individual's genotype for 3UTR122GA is GA, then the value assigned for that SNP for that individual is 1. If the genotype is AA, the value is 2. If the genotype is GG, then the value is 0. This was done for all 28 SNPs, and principal components analysis (PCA) was conducted using all 28 sets of values. The MMATI Stage 4 study group was used as a reference set for derivation of the principal component analysis (PCA). The scores of each individual in this group were plotted according to the statistical test (see FIG. 1). Based on the distribution, a cut-off of 0.126 on Component 2 (FIG. 1) was used to separate the study group into two sets. To demonstrate the survival significance, each individual was then plotted by the grouping >0.126 vs. <0.126 using the Kaplan-Meir method (FIG. 3). The same procedure was applied to other study groups using the exact same formulate as obtained from the reference group (MMAIT Stage 4). Moving forward, the same process is applicable to other individuals when utilizing the invention.

In specific embodiments, when one or more of the 28 SNPs fulfill a “treatment” score represented by a formula (and cutoff above 0.126) is detected, the individual may receive the cancer immunotherapy, but when one or more of the 28 SNPs does not fulfill a “treatment” score represented by the formula (and cutoff above 0.126), the individual may not receive the cancer immunotherapy.

### II. DIAGNOSIS OF MELANOMA

In some cases, an individual in need of being informed of responsiveness to a cancer immunotherapy has melanoma. The melanoma for which the immunotherapy may be utilized as treatment may have been diagnosed by visualization, biopsy, dermoscopic imaging, chest x-ray, lactate dehydrogenase test, CT, PET, MRI, or a combination thereof, for example.

### III. BACILLUS CALMETTE-GUÉRIN THERAPY

Bacillus Calmette-Guérin (BCG) (also referred to as TheraCys and TICE®) is an inactivated form of the tuberculosis bacteria that works against cancer as a biologic response modifier that elicits the immune system to indirectly affect tumors. In particular embodiments, BCG triggers a local inflammatory reaction that brings white blood cells and cytokines to the desired site. The immune system cells then fight directly against the tumor cells, and the cytokines change the tumor environment to inhibit future tumor growth. This is a test model for immunotherapy in melanoma.

### IV. SAMPLING

In order to assess the genetic make-up of an individual, it is necessary to obtain a nucleic acid-containing sample. Suitable tissues include almost any nucleic acid containing tissue, but those most convenient include oral tissue or blood. For those DNA specimens isolated from peripheral blood specimens, blood may be collected in heparinized/EDTA/sodium citrate anti-coagulant type tubes or anti-coagulant containing syringes or other appropriate vessel following venipuncture with a hypodermic needle. Oral tissue may advantagenously be obtained from a mouth rinse. Oral tissue or buccal cells may be collected with oral rinses or by swabbing of the oral cavity. Obtaining nucleic acid from tissue is routine in the art. Tissue may also can be obtained from normal tissue of patients from any organ.

### V. METHODS FOR DETECTION OF SNPS

The presence or absence of one or more of the SNPs of the invention may be evaluated using one or more of various techniques. For example, a gene may be cloned and sequenced to determine the presence or absence of a single nucleotide polymorphism. In certain embodiments, real-time PCR may be used to detect a single nucleotide polymorphism of the present invention. In other embodiments, techniques including PCR, multiplex PCR, gel electrophoresis, sequencing, hybridization with a probe specific for a single nucleotide polymorphism, restriction endonuclease digestion, primer extension, microarray or gene chip analysis, mass spectrometry, or a DNAse protection assay may be used for detecting a polymorphism of the present invention.

In some cases, one may employ MALDI-TOF mass spectrometry-based SNP genotyping (Sequenom®; San Diego, Calif.) or one may employ methodologies from Oxford Nanopore Technologies®, for example.

A skilled artisan recognizes that how one detects sequence variation (SNPs) is not necessarily important in the estimation of response to immunotherapy in melanoma; the key is the gene(s) and polymorphism(s) that one examines.

The following representative materials and methodologies are set forth as examples of the technology that can be applied in the context of the present invention.

A. Real-Time PCR (rtPCR)

The presence of absence of polymorphisms of the present invention may be detected using real-time PCR. Real-time PCR typically utilizes fluorescent probes for the selective detection of the polymorphisms. Various real-time PCR testing platforms that may be used with the present invention include: 5′ nuclease (TaqMan® probes), molecular beacons, and FRET hybridization probes. These detection methods rely on the transfer of light energy between two adjacent dye molecules, a process referred to as fluorescence resonance energy transfer (see, e.g., Espy et al (2006) Clin Microbiol Rev. 2006 January; 19(1): 165-256 for a review of various rtPCR approaches that may be used with the present invention).

1. 5′ Nuclease Probes

In certain embodiments, a 5′ nuclease probe may be used to detect a polymorphism of the present invention. 5′ nuclease probes are often referred to by the proprietary name, TaqMan® probes. A TaqMan® probe is a short oligonucleotide (DNA) that contains a 5′ fluorescent dye and 3′ quenching dye. To generate a light signal (i.e., remove the effects of the quenching dye on the fluorescent dye), two events must occur. First, the probe must bind to a complementary strand of DNA, e.g., at about 60° C. Second, at this temperature, Taq polymerase, which is commonly used for PCR, must cleave the 5′ end of the TaqMan® probe (5′ nuclease activity), separating the fluorescent dye from the quenching dye.

In order to differentiate a single nucleotide polymorphism from a wild-type sequence in the DNA from a subject, a second probe with complementary nucleotide(s) to the polymorphism and a fluorescent dye with a different emission spectrum are typically utilized. Thus, these probes can be used to detect a specific, predefined polymorphism under the probe in the PCR amplification product. Two reaction vessels are typically used, one with a complementary probe to detect wild-type target DNA and another for detection of a specific nucleic acid sequence of a mutant strain. Because TaqMan® probes typically require temperatures of about 60° C. for efficient 5′ nuclease activity, the PCR may be cycled between about 90-95° C. and about 60° C. for amplification. In addition, the cleaved (free) fluorescent dye can accumulate after each PCR temperature cycle; thus, the dye can be measured at any time during the PCR cycling, including the hybridization step. In contrast, molecular beacons and FRET hybridization probes typically involve the measurement of fluorescence during the hybridization step.

Genotyping for the SNPs of the invention may be evaluated using the following (5′ endonuclease probe) real-time PCR technique. Genotyping assays can be performed in duplicate and analyzed on a Bio-Rad iCycler Iq® Multicolor Real-time detection system (Bio-Rad Laboratories, Hercules, Calif.). Real-time polymerase chain reaction (PCR) allelic discrimination assays to detect the presence or absence of specific single nucleotide polymorphisms in the respective genes may utilize fluorogenic TaqMan® Probes.

Real-time PCR amplifications may be carried out in a 10 μl reaction mix containing 5 ng genomic DNA, 900 Nm of each primer, 200 Nm of each probe and 5 μl of 2× TaqMan® Universal PCR Master Mix (contains PCR buffer, passive reference dye ROX, deoxynucleotides, uridine, uracil-N-glycosylase and AmpliTaq Gold DNA polymerase; Perkin-Elmer, Applied Biosystems, Foster City, Calif.). Cycle parameters may be: 95° C. for 10 min, followed by 50 cycles of 92° C. for 15 sec and 60 C.° for 1 min. Real-time fluorescence detection can be performed during the 60° C. annealing/extension step of each cycle. The IQ software may be used to plot and automatically call genotypes based on a two parameter plot using fluorescence intensities of FAM and VIC at 49 cycles.

2. Molecular Beacons

Molecular beacons are another real-time PCR approach which may be used to identify the presence or absence of a polymorphism of the present invention. Molecular beacons are oligonucleotide probes that are labeled with a fluorescent dye (typically on the 5′ end) and a quencher dye (typically on the 3′ end). A region at each end of the molecular beacon probe is designed to be complementary to itself, so at low temperatures the ends anneal, creating a hairpin structure. This hairpin structure positions the two dyes in close proximity, quenching the fluorescence from the reporter dye. The central region of the probe is designed to be complementary to a region of a PCR amplification product. At higher temperatures, both the PCR amplification product and probe are single stranded. As the temperature of the PCR is lowered, the central region of the molecular beacon probe may bind to the PCR product and force the separation of the fluorescent reporter dye from the quenching dye. Without the quencher dye in close proximity, a light signal from the reporter dye can be detected. If no PCR amplification product is available for binding, the probe can re-anneal to itself, bringing the reporter dye and quencher dye into close proximity, thus preventing fluorescent signal.

Two or more molecular beacon probes with different reporter dyes may be used for detecting single nucleotide polymorphisms. For example, a first molecular beacon designed with a first reporter dye may be used to indicate the presence of a SNP and a second molecular beacon designed with a second reporter dye may be used to indicate the presence of the corresponding wild-type sequence; in this way, different signals from the first and/or second reporter dyes may be used to determine if a subject is heterozygous for a SNP, homozygous for a SNP, or homozygous wild-type at the corresponding DNA region. By selection of appropriate PCR temperatures and/or extension of the probe length, a molecular beacons may bind to a target PCR product when a nucleotide polymorphism is present but at a slight cost of reduced specificity. Molecular beacons advantageously do not require thermocycling, so temperature optimization of the PCR is simplified.

3. FRET Hybridization Probes

FRET hybridization probes, also referred to as LightCycler® probes, may also be used to detect a polymorphism of the present invention. FRET hybridization probes typically comprise two DNA probes designed to anneal next to each other in a head-to-tail configuration on the PCR product. Typically, the upstream probe has a fluorescent dye on the 3′ end and the downstream probe has an acceptor dye on the 5′ end. If both probes anneal to the target PCR product, fluorescence from the 3′ dye can be absorbed by the adjacent acceptor dye on the 5′ end of the second probe. As a result, the second dye is excited and can emit light at a third wavelength, which may be detected. If the two dyes do not come into close proximity in the absence of sufficient complimentary DNA, then FRET does not occur between the two dyes. The 3′ end of the second (downstream) probe may be phosphorylated to prevent it from being used as a primer by Taq during PCR amplification. The two probes may encompass a region of 40 to 50 DNA base pairs.

FRET hybridization probe technology permits melting curve analysis of the amplification product. If the temperature is slowly raised, probes annealing to the target PCR product will be reduced and the FRET signal will be lost. The temperature at which half the FRET signal is lost is referred to as the melting temperature of the probe system. A single nucleotide polymorphism in the target DNA under a hybridization FRET probe will still generate a signal, but the melting curve will display a lower Tm. The lowered Tₘ can indicate the presence of a specific polymorphism. The target PCR product is detected and the altered Tm informs the user there is a difference in the sequence being detected. Like molecular beacons, there is not a specific thermocycling temperature requirement for FRET hybridization probes. Like molecular beacons, FRET hybridization probes have the advantage of being recycled or conserved during PCR temperature cycling, and a fluorescent signal does not accumulate as PCR product accumulates after each PCR cycle.

B. Primer Extension

Primer extension is another technique which may be used according to the present invention. A primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present. For example, U.S. Ser. No. 10/407,846, which is which is hereby incorporated by reference, describes a form of primer extension.

Orchid BioSciences has a method called SNP-IT™ (SNP-Identification Technology) that uses primer extension with labeled nucleotide analogs to determine which nucleotide occurs at the position immediately 3′ of an oligonucleotide probe.

C. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

D. Sequencing

DNA sequencing may be used to evaluate a polymorphism of the present invention. For example, Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

E. Mass Spectrometry

Mass spectrometry may also be used to detect a polymorphism of the present invention. By exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). Methods of mass spectroscopy that may be used with the present invention include: ESI, ESI tandem mass spectroscopy (ESI/MS/MS), Secondary ion mass spectroscopy (SIMS), Laser desorption mass spectroscopy (LD-MS), Laser Desorption Laser Photoionization Mass Spectroscopy (LDLPMS), and MALDI-TOF-MS.

F. Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Sequenom uses a hybridization capture technology plus MALDI-TOF (Matrix Assisted Laser Desorption/Ionization-Time-of-Flight mass spectrometry) to detect sequence variation with their MassARRAY™ system (iPLEX assay).

Promega has the READIT™ SNP/Genotyping System (U.S. Pat. No. 6,159,693). In this method, DNA or RNA probes are hybridized to target nucleic acid sequences. Probes that are complementary to the target sequence at each base are depolymerized with a proprietary mixture of enzymes, while probes which differ from the target at the interrogation position remain intact. The method uses pyrophosphorylation chemistry in combination with luciferase detection to provide a highly sensitive and adaptable SNP scoring system.

G. Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments, as described above, also contemplate the use of Taqman® and Molecular Beacon® probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples may be detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P may be used. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

H. Amplifying a Target Sequence

In a particular embodiment, it may be desirable to amplify the target sequence before evaluating the SNP. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al., 1988, each of which is incorporated herein by reference in their entirety.

A reverse transcriptase PCR™ procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with XbaI, and ligate on M13-universal primers with an XbaI over hang, followed by amplification of the genomic DNA with an M13 universal primer. Only a small percentage of the total DNA would be amplified (the restriction fragments that were less than 1000 bases). One would then use labeled primers that correspond to a SNP are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

### I. OTHER EMBODIMENTS

Third Wave Technologies has the Invader OS™ method that uses their proprietary Cleavase® enzymes, which recognize and cut only the specific structure formed during the Invader process The Invader OS relies on linear amplification of the signal generated by the Invader process, rather than on exponential amplification of the target. The Invader OS assay does not utilize PCR in any part of the assay.

Finally, there are a number of forensic DNA testing labs and many research labs that use gene-specific PCR, followed by restriction endonuclease digestion and gel electrophoresis (or other size separation technology) to detect RFLPs in much the same way that the inventors have.

### VI. CHIPS

As discussed above, one convenient approach to detecting variation involves the use of nucleic acid arrays placed on chips. This technology has been widely exploited by companies such as Affymetrix, and a large number of patented technologies are available. Specifically contemplated are chip-based DNA technologies such as those described by Hacia et al. (1996) and Shoemaker et al. (1996). These techniques involve quantitative methods for analyzing large numbers of sequences rapidly and accurately. The technology capitalizes on the complementary binding properties of single stranded DNA to screen DNA samples by hybridization. Pease et al. (1994); Fodor et al. (1991).

Basically, a DNA array or gene chip consists of a solid substrate to which an array of single-stranded DNA molecules have been attached. For screening, the chip or array is contacted with a single-stranded DNA sample, which is allowed to hybridize under stringent conditions. The chip or array is then scanned to determine which probes have hybridized. In a particular embodiment of the instant invention, a gene chip or DNA array would comprise probes specific for chromosomal changes evidencing the predisposition towards the development of a neoplastic or preneoplastic phenotype. In the context of this embodiment, such probes could include PCR products amplified from patient DNA synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832, which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter, which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), and photopolymers (which contain photoreactive species such as nitrenes, carbenes and ketyl radicals) capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellose membranes, the DNA probes are spotted onto the membranes. A UV light source (Stratalinker™, Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

### VII. METHODS FOR NUCLEIC ACID SEPARATION

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized with light exhibiting the appropriate excitatory spectra.

### VIII. PERSONAL HISTORY MEASURES

In addition to use of the genetic analysis disclosed herein, the present invention makes use of additional factors in gauging an individual's prognosis for responding to BCG therapy or other immunomodulation therapy in an individual being treated or about to be treated therefore, such as for melanoma. In particular, one will examine multiple factors including age, sex, Breslow depth, ulceration, number of positive lymph nodes, primary site, ethnicity, smoking history, body mass index, alcohol consumption history, exercise history, and/or diet to improve the predictive accuracy of the present methods. A history of cancer in a relative, and the age at which the relative was diagnosed with cancer, are also useful personal history measures. The inclusion of personal history measures with genetic data in an analysis to predict a phenotype, response to cancer therapy in this case, is grounded in the realization that almost all phenotypes are derived from a dynamic interaction between an individual's genes and the environment in which these genes act. Those skilled in the art will realize that the personal history measures listed in this paragraph are unlikely to be the only such environmental factors that affect the penetrance of the cancer phenotype.

### IX. KITS OF INVENTION

Any of the compositions described herein may be comprised in a kit. The kits will thus comprise, in suitable container means, a therapeutic composition(s) and optionally an additional agent of the present invention. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the therapeutic composition and any other reagent containers in close confinement for commercial sale. Such containers may include injection or blow-molded plastic containers into which the desired vials are retained.

In some cases, the container means may itself be a syringe, pipette, and/or other such like apparatus, from which the formulation may be applied to an infected area of the body, injected into an animal, and/or even applied to and/or mixed with the other components of the kit.

The components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are included to demonstrate some embodiments of the invention. It should be appreciated by those of skill in the art that the techniques disclosed in the examples which follow represent techniques discovered by the inventor to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Methods

MMAIT-III was a Phase III prospective randomized international multicenter trial of BCG+melanoma vaccine vs. BCG+placebo after complete resection of Stage III and IV melanoma patients (NIH #NCT00052130). 361 patients (269 Stage III patients, 92 Stage IV patients) treated with resection and BCG+placebo had lymphocytes(PBL) from USA sites available for analysis. This served as the experimental group.

MSLT was a Phase III prospective randomized international multicenter trial of wide excision+regional lymph node observation (with lymphadenectomy at time of lymph node recurrence) vs. wide excision+sentinel lymph node biopsy (with immediate lymphadenectomy for metastasis detected on biopsy) of melanoma patients (NIH #NCT00275496). 80 patients (in either study group) had lymphocytes (PBL) from USA sites available for analysis. The selected patients were not treated with any immunomodulating therapy or chemotherapy. This served as the control group.

Endpoints for the SNP analysis were overall survival (OS) and disease-free survival(DFS), with a 10-yr follow-up. PBL DNA was assessed by MassARRAY MALDI-TOF for 28 SNPs associated with macrophage/monocyte-related immune response pathways to BCG and tuberculosis. Principal component analysis (PCA) utilizing MMAIT Stage 4 patients was performed, and a cutoff was identified by the PCA plot and used as a predictor in a Cox proportional hazard model.

### Example 2

### SNPS Associated with BCG Immunotherapy Responsiveness

The 28 SNPs had predictive value and distinguished patients in 2 survival groups (FIG. 3). In Cox model, the SNP panel was a significant predictor of survival independent of known stage melanoma prognostic factors.

The exemplary 28-SNP panel identified patients with an exceptionally favorable disease outcome in patients receiving BCG immunotherapy, and represents a stratifying predictive biomarker panel for identifying patients that are inherently responsive to BCG and other immunomodulating agents.

Table 1 shows the allele state of each of the 28 SNPs in the invention. Using the Stage IV MMAIT patients (BCG-treated group), principal components analysis (PCA) was conducted on the allele states of each 28 SNPs.*

| SNP RSID | Gene | Genotype |
|---|---|---|
| rs684 | ITGB2 (CD18, MAC1) | rs684: G/A |
| rs117989670 | ITGB2 (CD18, MAC1) | rs117989670: A/C |
| rs1135791 | SP110_Exon | rs1135791: C/T |
| rs1143627 | IL1B | rs1143627: C/T |
| rs1143634 | IL1B_Exon | rs1143634: C/T |
| rs1343151 | IL23R_Intron | rs1343151: C/T |
| rs16944 | IL1B | rs16944: A/G |
| rs17215556 | SLC11A1(NRAMP1)_Exon | rs17215556: C/T |
| rs17235409 | SLC11A1(NRAMP1)_Exon | rs17235409: A/G |
| rs17235416 | SLC11A1(NRAMP1) | rs17235416: —/TGTG |
| rs17860508 | IL12B | rs17860508: CTCTAA/GC |
| rs1799987 | CCR5 (CD195)_Intron | rs1799987: A/G |
| rs1800629 | TNF | rs1800629: A/G |
| rs1800896 | IL10 | rs1800896: A/G |
| rs1801157 | CXCL12 | rs1801157: A/G |
| rs2076530 | BTNL2_Exon | rs2076530: A/G |
| rs2457291 | ANKRD20A4 | rs2457291: A/T |
| rs2569190 | CD14_Intron | rs2569190: A/G |
| rs2569193 | CD14 | rs2569193: A/G |
| rs34448891 | SLC11A1(NRAMP1) | rs34448891: —/GT |
| rs361525 | TNF | rs361525: A/G |
| rs3731865 | SLC11A1(NRAMP1)_Intron | rs3731865: C/G |
| rs3751143 | P2X7_Exon | rs3751143: G/T |
| rs3948464 | SP110_Exon | rs3948464: C/T |
| rs4073 | IL8 | rs4073: A/T |
| rs4696480 | TLR2_Intron | rs4696480: A/T |
| rs4804803 | CD209 (DCSIGN) | rs4804803: A/G |
| rs5743708 | TLR2_Exon | rs5743708: A/G |

*e.g., For 3UTR122GA, the number of “A” alleles were counted—if the patient's genotype for 3UTR122GA is GA, then the value assigned for that SNP for that patient is 1. If the genotype is AA, the value is 2. If the genotype is GG, then the value is 0. This was done for all 28 SNPs, and PCA was conducted using all 28 sets of values. ** SNPs listed are based on the hg19 Human Genome Browser from the UCSC Bioinformatics site.

Table 2 shows the coefficients used for the calculation of principal component 2.

| SNP | Prin2 |
|---|---|
| 3UTR122GA-A | 0.14491 |
| 3UTR370GT-G | −0.00511 |
| rs1135791-C | 0.06345 |
| rs1143627-T | −0.52894 |
| rs1143634-C | 0.22158 |
| rs1343151-C | 0.21104 |
| rs16944-C | −0.52894 |
| rs17215556-C | −0.0021 |
| rs17235409-G | −0.01917 |
| rs17235416-TGTG | −0.02527 |
| rs17860508-GC | 0.00073 |
| rs1799987-A | 0.38151 |
| rs1800629-G | 0.09615 |
| rs1800896-A | 0.04412 |
| rs1801157-G | 0.06635 |
| rs2076530-A | 0.03816 |
| rs2457291-T | −0.02714 |
| rs2569190-G | 0.20832 |
| rs2569193-A | −0.01333 |
| rs34448891-GT | 0.0061 |
| rs361525-G | −0.02688 |
| rs3731865-C | 0.07898 |
| rs3751143-T | −0.23845 |
| rs3948464-C | −0.01395 |
| rs4073-A | 0.21229 |
| rs4696480-T | 0.04233 |
| rs4804803-A | 0.02972 |
| rs5743708-G | 0.04518 |

Table 3 shows multivariate Cox proportional hazards regression. The PCA grouping is shown to be an independent predictor of survival among MMAIT patients.

| | OS | | | | | | | |
|---|---|---|---|---|---|---|---|---|
| | (Univ) | | OS | | DFS | | DFS | |
| | p- | | (Multiv) | | (Univ) | | (Multiv) | |
| Factor | value | OS (Univ) HR | p-value | OS (Multiv) HR | p-value | DFS (Univ) HR | p-value | DFS (Multiv) HR |
| PCA Comp 2 | 0.0537 | 1.44 (0.99-2.10) | 0.0272 | 1.52 (1.05-2.22) | 0.0304 | 1.30 (1.03-1.66) | 0.0083 | 1.49 (1.11-2.01) |
| (MMVT4) | | | | | | | | |
| Age | 0.0256 | 1.02 (1.00-1.03) | 0.0697 | 1.01 (0.99-1.03) | 0.0131 | 1.01 (1.00-1.02) | 0.0892 | 1.82 (0.91-3.65) |
| Breslow | 0.2224 | | | | 0.2532 | | | |
| LN Positive | 0.0085 | 1.05 (1.01-1.08) | | | 0.0123 | 1.04 (1.01-1.07) | | |
| Primary Site | 0.7044 | | | | 0.6010 | | | |
| Gender | 0.0760 | | | | 0.1170 | | | |
| Group | 0.0101 | 1.92 (1.18-3.09) | 0.0117 | 2.18 (1.24-3.82) | <.0001 | 2.44 (1.70-3.52) | <.0001 | 2.91 (1.88-4.55) |
| (IIIP, IIINP, IV) | | | | | | | | |
| Ulceration | 0.0149 | 2.16 (1.26-3.77) | 0.0249 | 2.06 (1.20-3.60) | 0.0381 | 1.68 (1.09-2.61) | 0.0184 | 1.68 (1.09-2.61) |
| Overall Model P-value | | | 0.0003 | | | | <.0001 | |

*Multivariate: Stepwise Cox Regression eliminated #LN Positive; best overall model contains only four factors (Age, Treatment/Stage Group, Ulceration, and PCA Comp 2).

FIG. 1 shows results of principal components analysis (PCA) using the Stage IV MMAIT patients (BCG-treated group). The first two principal components are shown.

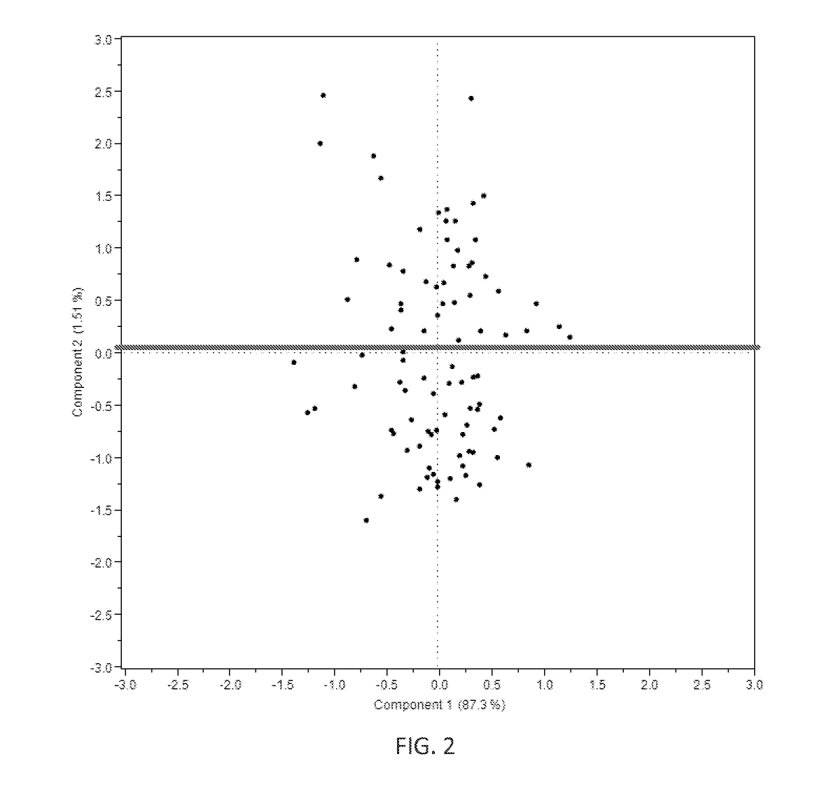

FIG. 2 shows results of PCA using the Stage IV MMAIT patients (BCG-treated group). The first two principal components are shown. A cutoff (red-coloured line) at the mean of component 2 was taken to separate patients into two groups: Group 1 is Comp2>0.126; Group2 is Comp2<=0.126

FIG. 3 shows application of formula (and cutoff above 0.126). A Kaplan-Meier curve was generated for the study patients, presented by AJCC Stage. OS=overall survival. DFS=disease-free survival. MMAIT=BCG-treated group. MSLT=non-treated group (i.e. control group).

### Example 3

### Identification of Responsiveness of an Individual to BCG Immunotherapy

In embodiments of the invention, an individual in need of BCG immunotherapy is subject to methods and/or compositions of the invention for determination of the presence of one or more SNPs indicative of responsiveness to BCG immunotherapy. In specific embodiments, the individual receiving the BCG immunotherapy treatment has cancer, such as melanoma. The individual may already be receiving the BCG immunotherapy upon analysis for one or more particular SNPs, or the individual may not yet be receiving the BCG immunotherapy upon analysis for one or more particular SNPs. An individual subjected to methods of the invention may have received, be receiving, or will be receiving an additional cancer therapy, such as surgery, radiation, chemotherapy, immunotherapy, and so forth.

A sample is obtained from an individual by standard means in the art. In specific cases, for example, a punch biopsy is obtained from an individual, nucleic acid is extracted from the cancer cells of the sample, the nucleic acid is optimally amplified, and the presence of SNPs are determined by standard means in the art. In some aspects, when one or more of the 28 SNPs fulfill a “treatment” score represented by the formula (and cutoff above 0.126) is detected, then the individual is responsive to BCG immunotherapy (or other immunomodulating agent(s)) and will have a favorable disease outcome. In some aspects, when one or more of the 28 SNPs does not fulfill a “treatment” score represented by the formula (and cutoff above 0.126) then the individual will not be responsive to BCG immunotherapy (or other immunomodulating agent(s)), and a medical provider may opt not to use BCG immunotherapy (or other immunomodulating agent(s)) for the individual.

However, in some embodiments, when one or more of the SNPs in is present and/or in a specific allelic configuration of NRAMP1, CD14, CD18 (MAC1), CD195 (CCR5), CD209 (DC-SIGN), CD282 (TLR2), IL1, IL10, IL12, IL23, TNF, P2X7, IL-8, and/or CXCL12, then the individual will be responsive to BCG immunotherapy (or other immunomodulating agent(s)) and will have a favorable disease outcome. When none of the SNPs are identified, then the individual will not be responsive to BCG immunotherapy, or other immunomodulating agent, and a medical provide may opt not to use BCG immunotherapy (and/or other immunomodulating agent) for the individual.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PATENTS AND PATENT APPLICATIONS

U.S. Pat. No. 5,843,663
U.S. Pat. No. 5,900,481
U.S. Pat. No. 5,919,626
U.S. Pat. No. 5,849,481
U.S. Pat. No. 5,849,486
U.S. Pat. No. 5,851,772
U.S. Pat. No. 6,159,693
U.S. Pat. No. 4,683,195
U.S. Pat. No. 4,683,202
U.S. Pat. No. 4,800,159
U.S. Pat. No. 5,882,864
U.S. Pat. No. 5,843,650
U.S. Pat. No. 5,846,709
U.S. Pat. No. 5,846,783
U.S. Pat. No. 5,849,546
U.S. Pat. No. 5,849,497
U.S. Pat. No. 5,849,547
U.S. Pat. No. 5,858,652
U.S. Pat. No. 5,866,366
U.S. Pat. No. 5,916,776
U.S. Pat. No. 5,922,574
U.S. Pat. No. 5,928,905
U.S. Pat. No. 5,928,906
U.S. Pat. No. 5,932,451
U.S. Pat. No. 5,935,825
U.S. Pat. No. 5,939,291
U.S. Pat. No. 5,942,391
U.S. Pat. No. 5,861,242
U.S. Pat. No. 5,578,832
GB Application No. 2 202 328
PCT Application No. PCT/US89/01025,
PCT Application WO 89/06700

### PUBLICATIONS

MMAIT: Morton D L, Mozzillo N, Thompson J F, Kelley M C, Faries M, Wagner J, Schneebaum S, Schuchter L, Gammon G, Elashoff R, MMAIT Clinical Trials Group I: An international, randomized, phase III trial of Bacillus Calmette-Guerin (BCG) plus allogeneic melanoma vaccine (MCV) or placebo after complete resection of melanoma metastatic to regional or distant sites. J Clin Oncol 25(18S): Abstract 8508, 2007.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of determining whether or not an individual will be responsive to immunotherapy, comprising the step of providing a representative value for a specific allelic configuration in one or more single nucleotide polymorphisms (SNPs) in nucleic acid from a sample from the individual and comparing the representative value to a pre-determined threshold value, wherein when the representative value is greater than the threshold, the individual will respond to the immunotherapy, wherein the SNPs are located in ITGB2, SP110, IL1B, IL23R, SLC11A1, IL12B, CCR5, TNF, IL10, CXCL12, BTNL2, ANKRD20A4, CD14, P2X7, IL8, TLR2, and/or CD209.

2. The method of claim 1, wherein the SNP is at least one of the SNPs identified in Table 1.

3. The method of claim 1, wherein the SNPs are all of the SNPs identified in Table 1.

4. The method of claim 1, wherein the immunotherapy comprises Bacillus Calmette-Guérin (BCG), cytokine therapy, monoclonal antibody-based therapy, anti-PD1/PD1-ligand therapy, MAPKinase inhibitor, anti-toll like receptor (TLR) therapy, dabrafenib, tumor-infiltrating lymphocyte therapies, or a combination thereof.

5. The method of claim 4, wherein the cytokine therapy comprises interferon (IFN), interleukin-2 (IL-2) therapy, or a combination thereof.

6. The method of claim 4, wherein the monoclonal antibody-based treatment comprises ipilimumab, vemurafenib, sorafenib, or a combination thereof.

7. The method of claim 1, wherein the individual has melanoma.

8. The method of claim 1, wherein the individual has received or is receiving a melanoma therapy.

9. The method of claim 8, wherein the melanoma therapy comprises surgery, radiation, chemotherapy, or immunotherapy.

10. The method of claim 1, wherein the individual is human.

11. The method of claim 1, wherein the sample is tissue biopsy, serum, or blood.

12. The method of claim 1, further comprising the step of obtaining the sample from the individual.

13. The method of claim 1, wherein DNA is isolated from the sample.

14. The method of claim 1, wherein RNA is isolated from the sample.

15. The method of claim 1, wherein at least part of the nucleic acid from the sample is amplified prior to detection.

16. A method of treating an individual for cancer, wherein the cancer treatment comprises immunotherapy, comprising the step of providing an effective amount of the immunotherapy to the individual when a representative value for a specific allelic configuration in one or more single nucleotide polymorphisms (SNPs) in nucleic acid from a sample from the individual is greater than a pre-determined threshold value, wherein the SNPs are in genes selected from the group consisting of ITGB2, SP110, IL1B, IL23R, SLC11A1, IL12B, CCR5, TNF, IL10, CXCL12, BTNL2, ANKRD20A4, CD14, P2X7, IL8, TLR2, CD209; and a combination thereof.

17. The method of claim 16, wherein the SNP is at least one of the SNPs identified in Table 1.

18. The method of claim 16, wherein the SNPs are all of the SNPs identified in Table 1.

19. The method of claim 16, wherein the immunotherapy comprises Bacillus Calmette-Guérin (BCG), cytokine therapy, monoclonal antibody-based therapy, anti-PD1/PD1-ligand therapy, MAPKinase inhibitor, anti-TLR therapy, or a combination thereof.

20. The method of claim 16, further comprising the step of providing an anti-cancer agent to the individual.

21. The method of claim 16, further comprising the step of identifying an individual in need of the cancer treatment.

22. The method of claim 16, wherein the cancer is melanoma.

23. The method of claim 22, wherein the individual has had surgical removal of at least part of the melanoma.
